(19) United States
(12) Patent Application Publication
(10) Pub. No.: US 2021/0128521 A1
(43) Pub. Date: May 6, 2021

(54) METHODS OF USING CANNABINOID COMPOSITIONS IN SPORTS MEDICINE APPLICATIONS

(71) Applicant: Precision Biologics, Marietta, GA (US)

(72) Inventor: Peyton Palaio, Denver, CO (US)

(21) Appl. No.: 17/082,915

(22) Filed: Oct. 28, 2020

Related U.S. Application Data
(60) Provisional application No. 62/928,206, filed on Oct. 30, 2019.

(51) Int. Cl. A61K 31/352; A61K 31/05; A61K 9/00; A61K 47/44; A61K 47/22

(52) U.S. Cl. CPC A61K 31/352; A61K 31/05; A61K 47/22; A61K 47/44; A61K 9/0014

(57) ABSTRACT

Provided are compositions comprising a cannabinoid, an oil (e.g., MCT oil or coconut oil), and a wax, and methods of making such compositions. The disclosed compositions can be useful in, for example, treating an injury of the muscle, joint, cartilage, and/or tendon. This abstract is intended as a scanning tool for purposes of searching in the particular art and is not intended to be limiting of the present invention.

### CROSS-REFERENCE TO RELATED APPLICATIONS

This Application claims the benefit of U.S. Provisional Application No. 62/928,206, filed on Oct. 30, 2019, the contents of which are incorporated herein by reference in their entirety.

### BACKGROUND

Pain represents a significant public health issue. According to the National Institutes of Health (NIH), pain affects more Americans than diabetes, heart disease, and cancer combined. Further, pain is cited as the most common reason Americans access the health care system, and chronic pain is the most common cause of long-term disability. Despite this high prevalence, the underlying etiology and mechanisms of pain can be quite complex. In general, pain can be separated into four categories: somatic pain, visceral pain, neuropathic pain, and psychogenic pain. Somatic pain is caused by the activation of pain receptors in either the body surface or musculoskeletal tissues. Visceral pain is the pain derived from damage or injury to the internal organs. Neuropathic pain is caused by injury to or malfunction of the spinal cord and peripheral nerves. Finally, psychogenic pain is physical pain that is caused, increased, or prolonged by mental, emotional, or behavioral factors. In addition, pain can be acute or chronic. Each of these distinctions further complicate the understanding of this disorder and, in turn, further complicate the available treatments.

Examples of medications that have been used to treat pain include non-steroidal anti-inflammatory drugs (NSAIDs), acetaminophen, antidepressants, anti-seizure medicines, steroids, opioids, analgesics, and antihistamines. Unfortunately, these drugs are not always safe, effective, or even appropriate. As such, there remains a significant unmet need for improved treatment methods. This unmet need is particularly true for patients suffering from neuropathic pain. For example, up to 50% of patients with neuropathic pain do not respond to any treatment at all.

Cannabinoids are a class of compounds that act on the cannabinoid receptors in cells. Cannabinoids can be endogenous (i.e., endocannabinoids; present endogenously in human or animal tissues), synthetic, or derived from plants (i.e., phytocannabinoids). A well-known source of phytocannabinoids is the genus of plants known as Cannabis or more colloquially referred to as marijuana. At least 104 phytocannabinoids have been isolated from marijuana to date, and many of these compounds have widely variable biological activities and properties. Cannabinoids have been reported to provide various therapeutic benefits for humans, including effects that are anti-psychotic, analgesic, anti-inflammatory, anti-spasmodic, anti-convulsant, anti-emetic, antioxidant, neuroprotective, and immunomodulatory. In addition, certain cannabinoids have been investigated for their benefit in treating symptoms of pain; however, there are significant challenges and issues relating to their safe, effective, and federally acceptable use such as, for example, a wide range of side effects, a complex mechanisms of action, purity, consistency, quality control, product sourcing, and cost.

Thus, despite the potential benefits of using cannabinoids in the treatment of pain, the safe and effective incorporation of cannabinoids into pain-relief compositions has remained elusive. Thus, there remains a need for cannabinoid compositions and methods of making and using same. These needs and others are met by the present invention.

### SUMMARY

In accordance with the purpose(s) of the invention, as embodied and broadly described herein, the invention, in one aspect, relates to topical cannabinoid compositions and methods of making and using same in, for example, the treatment of injuries involving the muscles, joints, cartilage, and/or tendons.

Thus, disclosed are compositions comprising: (a) a cannabinoid in an amount of from about 10 wt % to about 14 wt %; (b) an oil; and (c) a wax.

Also disclosed are compositions comprising: (a) a cannabinoid in an amount of from about 10 wt % to about 14 wt %; (b) an oil selected from MCT oil and coconut oil; and (c) a wax.

Also disclosed are methods for making a disclosed composition.

Also disclosed are methods for treating an injury on a subject, the method comprising topically administering to the wound an effective amount of a disclosed composition, wherein the injury is a muscle injury, a joint injury, a cartilage injury, a tendon injury, or a combination thereof.

Also disclosed are kits comprising a disclosed composition, and one or more of: (a) an agent known for the treatment of pain; (b) instructions for treating an injury selected from a muscle injury, a joint injury, a cartilage injury, a tendon injury, or a combination thereof, wherein the composition and the agent are not co-formulated.

Still other objects and advantages of the present disclosure will become readily apparent by those skilled in the art from the following detailed description, wherein it is shown and described only the preferred aspects, simply by way of illustration of the best mode. As will be realized, the disclosure is capable of other and different aspects, and its several details are capable of modifications in various obvious respects, without departing from the disclosure. Accordingly, the description is to be regarded as illustrative in nature and not as restrictive.

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present compositions, methods and systems may be understood more readily by reference to the following detailed description of preferred embodiments and the examples included therein and to the Figures and their previous and following description.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and the appended claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

As used herein, the term “pharmaceutically acceptable topical carrier” refers to a material, composition, diluent, or vehicle that is suitable for application to skin or mucosal surfaces, without undue toxicity, irritation, or allergic response. Examples of pharmaceutically acceptable topical carriers include, but are not limited to, creams, lotions, ointments, pastes, jellies, and gels. In various aspects, the pharmaceutically acceptable topical carrier is known as being useful in cosmetic agents and toiletry agents such as, for example, sunscreen and other sun products, anti-aging agents, moisturizing agents, and baby creams.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with an ailment, disease, or disorder. The term “patient” includes human and veterinary subjects.

As used herein, the term “treatment” refers to the medical management of a patient with the intent to cure, ameliorate, stabilize, or prevent an ailment, disease, pathological condition, disorder, or injury. This term includes active treatment, that is, treatment directed specifically toward the improvement of a skin ailment, disease, pathological condition, disorder, or injury, and also includes causal treatment, that is, treatment directed toward removal of the cause of the associated skin ailment, disease, pathological condition, disorder, or injury. In addition, this term includes palliative treatment, that is, treatment designed for the relief of symptoms rather than the curing of the skin ailment, disease, pathological condition, disorder, or injury; preventative treatment, that is, treatment directed to minimizing or partially or completely inhibiting the development of the associated skin ailment, disease, pathological condition, disorder, or injury; and supportive treatment, that is, treatment employed to supplement another specific therapy directed toward the improvement of the associated skin ailment, disease, pathological condition, disorder, or injury. In various aspects, the term covers any treatment of a subject, including a mammal (e.g., a human), and includes: (i) preventing the injury from occurring in a subject that can be predisposed to the injury but has not yet been diagnosed as having it; (ii) inhibiting the injury, i.e., arresting its development or exacerbation thereof; or (iii) relieving the injury, i.e., promoting healing of the injury. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compositions or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

“Topical” as used with respect to a composition means a composition that is applied to the skin or mucosal membrane of a subject such as, for example, a human. A topical composition is intended to have an effect at the site of application, i.e., in the tissue beneath the site of application, and does not result in significant drug concentrations in the blood and other tissues. In various aspects, a topical composition can be transdermal, i.e., absorbed through the skin/mucosal membrane and/or having a systemic effect in areas of the body away from the site of application. In various further aspects, a topical composition can be non-transdermal, i.e., having a local or non-systemic effect.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed composition or a product of a disclosed method of making, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed composition or a product of a disclosed method of making, suspended in sterile saline solution for injection together with a preservative.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition) , and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; anti-HIV agents such as entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “essentially,” in, for example, the context “essentially absent” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

Disclosed are also components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

### B. Compositions

In one aspect, disclosed are compositions comprising: (a) a cannabinoid in an amount of from about 10 wt % to about 14 wt %; (b) an oil; and (c) a wax.

In one aspect, disclosed are compositions comprising: (a) a cannabinoid in an amount of from about 10 wt % to about 14 wt %; (b) an oil selected from MCT oil and coconut oil; and (c) a wax.

In various aspects, the composition consists essentially of the cannabinoid, the oil, and the wax.

In various aspects, the composition is topical.

In various aspects, compositions of the present invention can be in any form suitable for topical use such as, for example, an aerosol, cream, ointment, lotion, dusting powder, paste, spray, lotion, gel, jelly, and the like. These formulations can be prepared via conventional processing methods known to one skilled in the art.

In a further aspect, the composition comprises an oil. Examples of oils include, but are not limited to, olive oil, almond oil, apricot kernel oil, avocado oil, borage seed oil, camellia seed oil (tea oil), cranberry seed oil, evening primrose oil, grapeseed oil, hazelnut oil, hemp seed oil, jojoba, kukui nut oil, macademia nut oil, meadowfoam oil, peanut oil, pecan oil, pomegranate seed oil, rose hip oil, seabuckthorn berry oil, sesame seed oil, coconut oil, sunflower oil, watermelon seed oil, and MCT oil, or mixtures thereof. Without wishing to be bound by theory, the amount of oil present can be dependent upon the formulation of the composition. For example, in various aspects, the oil is present in an amount of from about 20 wt % to about 30 wt %, 25 wt % to about 30 wt %, or 20 wt % to about 25 wt %. In various further aspects, the oil is present in an amount of from about 50 wt % to about 98 wt %, 60 wt % to about 98 wt %, 70 wt % to about 98 wt %, 80 wt % to about 98 wt %, 90 wt % to about 98 wt %, 50 wt % to about 90 wt %, 50 wt % to about 80 wt %, 50 wt % to about 70 wt %, or 50 wt% to about 60 wt %.

In various aspects, the oil is naturally occurring. In a still further aspect, the oil is non-naturally occurring (e.g., MCT oil).

In various aspects, the composition comprises an oil selected from MCT oil and coconut oil. In a further aspect, the oil is MCT oil. In a still further aspect, the oil is coconut oil.

In various aspects, the composition comprises a cannabinoid. Cannabinoids are a class of diverse chemical compounds that act on cannabinoid receptors on cells that repress neurotransmitter release in the brain. Cannabinoid receptors are of a class of cell membrane receptors under the G protein-coupled receptor superfamily. As is typical of G protein-coupled receptors, the cannabinoid receptors contain seven transmembrane spanning domains. There are currently two known subtypes of cannabinoid receptors, termed CB1 and CB2, with mounting evidence of more. The CB1 receptor is expressed mainly in the brain (central nervous system), but also in the lungs, liver and kidneys. The CB2 receptor is expressed mainly in the immune system and in hematopoietic cells.

The classical cannabinoids are derived from their respective 2-carboxylic acids (2-COOH) by decarboxylation, catalyzed by heat, light, or alkaline conditions. Phytocannabinoids (those derived from the Cannabis plant) include but not limited to: tetrahydrocannabinol (THC), tetrahydrocannabinolic acid (THCA), cannabidiolic acid (CBDA), cannabidiol (CBD), cannabinol (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV) and cannabigerol monomethyl ether (CBGM). Thus, in various aspects, the cannabinoid is THC, CBD, THCA, CBDA, CBN, CBG, CBC, or CBGM, or a mixture thereof. In a further aspect, the cannabinoid is THC or CBD, or a mixture thereof. In a still further aspect, the cannabinoid is THC. In yet a further aspect, the cannabinoid is CBD.

In a further aspect, the cannabinoid is present in an amount of from about 10 wt % to about 14 wt %. Thus, in various aspects, the cannabinoid is present in an amount of from about 10 wt % to about 13 wt %, about 10 wt % to about 12 wt %, about 10 wt % to about 11 wt %, about 11 wt % to about 14 wt %, about 12 wt % to about 14 wt %, about 13 wt % to about 14 wt %, or about 11 wt % to about 13 wt %.

In a further aspect, the composition further comprises vitamin E.

In a further aspect, the composition comprises vitamin E in an amount of from about 0.25 wt % to about 4 wt %, about 0.25 wt % to about 3 wt %, about 0.25 wt % to about 2 wt %, about 0.25 wt % to about 1 wt %, about 0.25 wt % to about 0.75 wt %, about 0.75 wt % to about 4 wt %, about 1 wt % to about 4 wt %, about 2 wt % to about 4 wt %, or about 3 wt % to about 4 wt %.

In a further aspect, the composition further comprises one or more of cocoa butter, shea butter, an essential oil, and a jojoba oil. In a still further aspect, the composition further comprises two or more of cocoa butter, shea butter, an essential oil, and a jojoba oil. In yet a further aspect, the composition further comprises three or more of cocoa butter, shea butter, an essential oil, and a jojoba oil. In an even further aspect, the composition further comprises each of cocoa butter, shea butter, an essential oil, and a jojoba oil. In a still further aspect, the composition further comprises cocoa butter. In yet a further aspect, the composition further comprises shea butter. In an even further aspect, the composition further comprises jojoba oil.

In various aspects, vitamin B12 is essentially absent from the composition. In a further aspect, vitamin B12 is present in the composition in an amount less than less than about 10 wt %, less than about 5 wt %, less than about 1 wt %, less than about 0.5 wt %, less than about 0.1 wt %, less than about 0.05 wt% , or less than about 0.01 wt %.

In various aspects, a nonsteroidal anti-inflammatory drug (NSAID) is essentially absent from the composition. In a further aspect, a NSAID is present in the composition in an amount less than less than about 10 wt %, less than about 5 wt %, less than about 1 wt %, less than about 0.5 wt %, less than about 0.1 wt %, less than about 0.05 wt %, or less than about 0.01 wt %.

In a further aspect, the composition further comprises an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others.

In a further aspect, the additive is present in an amount of from about 0.01 wt % to about 10 wt % of the composition. In a still further aspect, the additive is present in an amount of from about 0.01 wt % to about 8 wt % of the composition. In yet a further aspect, the additive is present in an amount of from about 0.01 wt % to about 6 wt % of the composition. In an even further aspect, the additive is present in an amount of from about 0.01 wt % to about 4 wt % of the composition. In a still further aspect, the additive is present in an amount of from about 0.01 wt % to about 2 wt % of the composition. In yet a further aspect, the additive is present in an amount of from about 0.01 wt % to about 1 wt % of the composition. In an even further aspect, the additive is present in an amount of from about 0.1 wt % to about 10 wt % of the composition. In a still further aspect, the additive is present in an amount of from about 1 wt % to about 10 wt % of the composition. In yet a further aspect, the additive is present in an amount of from about 2 wt % to about 10 wt % of the composition. In an even further aspect, the additive is present in an amount of from about 4 wt % to about 10 wt % of the composition. In a still further aspect, the additive is present in an amount of from about 6 wt % to about 10 wt % of the composition. In yet a further aspect, the additive is present in an amount of from about 8 wt % to about 10 wt % of the composition.

It is understood that the disclosed compositions can be employed in the disclosed methods of using.
1. Cannabinoids

For many years it was assumed that the chemical components of the cannabis plant, cannabinoids, produce analgesia by activating specific receptors throughout the body, in particular cannabinoid receptor 1 (CB1), which are found predominantly in the central nervous system (CNS), and cannabinoid receptor 2 (CB2), found predominantly in cells involved with immune function. However, recently this picture has become much more complicated, as it has been recognized that cannabinoids, both plant-derived and endogenous, act simultaneously on multiple pain targets within the peripheral and CNS. Specifically, besides acting on cannabinoid CB1/CB2 receptors, they may reduce pain through interaction with the putative non-CB1/CB2 cannabinoid G protein-coupled receptor (GPCR) 55 or GPCR 18 (also known as the N-arachidonoyl glycine (NAGly) receptor), and other well-known GPCRs, such as the opioid or serotonin (5-HT) receptors. In addition, many studies have reported the ability of certain cannabinoids to modulate nuclear receptors (peroxisome proliferator-activated receptors (PPARs), cys loop ligand-gated ion channels or transient receptor potential (TRP) channels (TRPV, TRPA, and TRPM subfamilies), among others. Moreover, TRPV1 and CB1 or CB2 are co-localized at peripheral and/or central neurons (sensory neurons, dorsal root ganglia, spinal cord, brain neurons), which results in their intracellular crosstalk in situations where these receptors are involved simultaneously. New data also demonstrate a variety of interactions between cannabinoid, opioid, and TRPV1 receptors in pain modulation. Without wishing to be bound by theory, these receptors represent potentially attractive targets for the therapeutic use of cannabinoids in the treatment of pain.
a. Phytocannabinoids

There are about 100 different cannabinoids isolated from the cannabis plant. The main psychoactive compound is THC, which is produced mainly in the flowers and leaves of the plant. The THC content varies from about 5% in marijuana to about 80% in hashish oil. THC is an analog to the endogenous cannabinoid, anandamide (ananda is the Sanskrit word for bliss; arachidonoylethanolamide, AEA). It is responsible for most of the pharmacological actions of cannabis, including the psychoactive, analgesic, anti-inflammatory, anti-oxidant, antipruritic, bronchodilatory, anti-spasmodic, and muscle-relaxant activities. THC acts as a partial agonist at cannabinoid receptors (CB1 and CB2). A very high binding affinity of THC with the CB1 receptor appears to mediate its psychoactive properties (changes in mood or consciousness), memory processing, motor control, etc. It has been reported that a number of side effects of THC, including anxiety, impaired memory and immunosuppression, can be reversed by other constituents of the cannabis plant (cannabinoids, terpenoids, and flavonoids).

The non-psychoactive analog of THC, cannabidiol (CBD), is another important cannabinoid found in the cannabis plant. It is thought to have significant analgesic, anti-inflammatory, anti-convulsant and anxiolytic activities without the psychoactive effect of THC. CBD has little binding affinity for either CB1 or CB2 receptors, but it is capable of antagonizing them in the presence of THC. In fact, CBD behaves as a non-competitive negative allosteric modulator of CB1 receptor, and it reduces the efficacy and potency of THC and AEA. CBD also regulates the perception of pain by affecting the activity of a significant number of other targets, including non-cannabinoid GPCRs (e.g., 5-HT1A), ion channels (TRPV1, TRPA1 and TPRM8, GlyR), PPARs, while also inhibiting uptake of AEA and weakly inhibiting its hydrolysis by the enzyme fatty acid amide hydrolase (FAAH). It has been demonstrated that cannabidiol can act synergistically with THC and contribute to the analgesic effect of medicinal-based cannabis extract. At the same time, CBD displays an entourage effect (the mechanism by which non-psychoactive compounds present in cannabis modulate the overall effects of the plant), and is capable of improving tolerability and perhaps also the safety of THC by reducing the likelihood of psychoactive effects and antagonizing several other adverse effects of THC (sedation, tachycardia, and anxiety). The differences in concentration of THC and CBD in the plant reflect the differences in the effects of different cannabis strains. Although CBD as a monotherapy in the treatment of pain has not been evaluated clinically, its anti-inflammatory and anti-spasmodic benefits and good safety profile suggest that it could be an effective and safe analgesic.

Other phytocannabinoids that can contribute to the overall analgesic effects of medical cannabis are cannabichromene (CBC), cannabigerol (CBG), tetrahydrocannabivarin (THCV), and many others. Similarly to CBD, these compounds do not display significant affinities for cannabinoid receptors, but they have other modes of action.
b. Endocannabinoids

The endocannabinoid system regulate many functions in the body, including learning and memory, mood and anxiety, drug addiction, feeding behavior, perception, modulation of pain and cardiovascular functions. The endocannabinoid system consists of cannabinoid receptors, endogenous cannabinoids (endocannabinoids), transport proteins and enzymes that synthesize or degrade the endocannabinoids.

Cannabinoid CB1 and CB2 receptors are 7-transmembrane G-protein coupled receptors (GPCRs). They play an important role in peripheral, spinal, and supraspinal nociception, including ascendant and descendent pain pathways. The signal transduction pathway of CB1 and CB2 involves inhibition of adenylyl cyclase, decreased cAMP formation, as well as an increase in the activity of mitogen-activated protein kinases (MAPK). Without wishing to be bound by theory, studies indicate that different ligands can differentially activate these pathways, suggesting biased signaling through the cannabinoid receptors CB1 and CB2.

The CB1 receptor is distributed throughout the nervous system. It mediates psychoactivity, pain regulation, memory processing, and motor control. CB1 is a presynaptic heteroreceptor that modulates neurotransmitter and neuropeptide release and inhibits synaptic transmission. Activation of CB1 results in the activation of inwardly rectifying potassium channels, which decrease presynaptic neuron firing, and in the inhibition of voltage-sensitive calcium channels that decrease neurotransmitter release. The CB1 receptor is strategically located in regions of the peripheral and CNS where pain signaling is intricately controlled, including the peripheral and central terminals of primary afferent neurons, the dorsal root ganglion (DRG), the dorsal horn of the spinal cord, the periaqueductal gray matter, the ventral posterolateral thalamus and cortical regions associated with central pain processing, including the anterior cingulate cortex, amygdala and prefrontal cortex. The principal endogenous ligand for the CB1 receptor is AEA. CB1 receptors are observed more often on the gamma-aminobutyric acid (GABA) inhibitory interneurons in the dorsal horn of the spinal cord, and weakly expressed in most excitatory neurons. CB1 receptors are also present in multiple immune cells such as macrophages, mast cells and epidermal keratinocytes.

The CB2 receptor is found predominantly at the periphery (in tissues and cells of the immune system, hematopoietic cells, bone, liver, peripheral nerve terminals, keratinocytes), but also in brain microglia. The receptors are responsible for the inhibition of cytokine/chemokine release and neutrophil and macrophage migration and they contribute to slowing down of chronic inflammatory processes and modulate chronic pain. Both CB2 and CB1 receptors on mast cells participate in the anti-inflammatory mechanism of action of cannabinoids. Also, activation of CB2 receptors on keratinocytes stimulates the release of β-endorphin, which acts at opioid receptors on peripheral sensory neurons to inhibit nociception. Under basal conditions, CB2 receptors are present at low levels in the brain, the spinal cord and DRG, but may be upregulated in microglia where they modulate neuroimmune interaction in inflammation and after peripheral nerve damage. CB2 receptor activation inhibits adenylyl cyclase activity and stimulates MAPK activity, but the effect on calcium or potassium conductance is controversial. Stimulation of CB2 receptors does not produce cannabis-like effects on the psyche and circulation. The principal endogenous ligand for the CB2 receptor is 2-arachidonoylglycerol (2-AG).

Endocannabinoids are arachidonic acid derivatives. AEA and 2-AG are synthesized separately, they have local (autocrine and paracrine) effects and are rapidly removed by hydrolysis by fatty acid amide hydrolase (FAAH) and monoacylglycerol lipase (MAGL), respectively. Besides AEA, FAAH inhibition significantly elevates the levels of other fatty-acid amides such as oleoylethanolamide (OEA) and palmitoylethanolamide (PEA) in the CNS and peripheral tissues. Endocannabinoids, similarly to THC, appear to activate cannabinoid receptors. AEA and 2-AG are a partial and full agonist of CB receptors, respectively. They work as a part of a negative feedback loop that regulates neurotransmitter and neuropeptide release and thereby modulate various CNS functions, including pain processing.

The AEA is a full agonist at TRPV1 (AEA referred to as an ‘endovanilloid’) that activates TRPV1 which results in desensitization. AEA also activates GR55, directly inhibits 5-HT3A receptors, potentiates the function of glycine receptors, inhibits T-type voltage-gated calcium channels, and activates PPARs.

Endocannabinoids, which are produced in neural and non-neural cells in the physiological response to tissue injury or excessive nociceptive signaling, suppress inflammation, sensitization and pain. Inhibitors of FAAH lead to elevated AEA levels and are intended for therapeutic use. N-acylethanolamines such as PEA and OEA do not belong to endocannabinoids as they do not bind to cannabinoid receptors; they exhibit anti-inflammatory action via PPARs, and also inhibit pain through TRPV1 receptors. They are also of interest to the field of cannabinoid pain research as they elevate levels of AEA through substrate competition at FAAH.

There is a constant active exchange of substrates and metabolites between endocannabinoid and eicosanoid pathways. The enzyme FAAH breaks down AEA to arachidonic acid and ethanolamine or, alternatively, AEA can be directly transformed by cyclooxygenase-2 (COX-2) into proalgesic prostaglandins. As such, AEA may contribute to the analgesic properties of COX-2 selective NSAIDs. It was established that the metabolite of paracetamol combines with arachidonic acid by the action of FAAH to produce an endocannabinoid, which is a potent agonist at the TRPV1 and a weak agonist at both CB1 and CB2 receptors and an inhibitor of AEA reuptake.
c. Synthetic Cannabinoids

At present, there are two synthetic cannabinoids on the market, dronabinol and nabilone, which may be of benefit in the treatment of pain. In general, their use in pain treatment is off-label. Dronabinol is a generic name for the oral form of synthetic THC (Marinol®). It is approved for the treatment of chemotherapy-associated nausea and vomiting, and anorexia associated with human immunodeficiency virus infection. Nabilone, a generic name for the orally administered synthetic structural analog of THC (Cesamet®), is approved for the treatment of chemotherapy-associated nausea and vomiting. Their medical use is mostly limited by their psychoactive side effects, as well as their limited bioavailability).
d. Cannabis and Cannabis Extract

Cannabis delivered by way of inhalation (smoked or inhaled through vaporization), orally or oromucosally, produces a host of biological effects. Unfortunately, clinical trials conducted on cannabis are limited, and no drug agency has approved the use of cannabis as a treatment for any medical condition. Although there is no formal approval, cannabis is widely used for the treatment of pain. It is authorized by physicians where medical marijuana is legal.

Nabiximols, a generic name for the whole-plant extract with a 1:1 ratio of THC:CBD (2.7 THC+2.5 CBD per 100 μL), an oromucosal spray (Sativex®) is approved as an adjuvant treatment for symptomatic relief of spasticity in adult patients with multiple sclerosis (MS) who have not responded well to other therapy and who have demonstrated a significant improvement during an initial trial of Sativex® therapy. In addition, Sativex® is approved in Canada (under the Notice of Compliance with Conditions) as an adjuvant treatment for symptomatic relief of neuropathic pain in adults with MS, and as an adjuvant analgesic in adult patients with advanced cancer who suffer from moderate to severe pain that is resistant to strong opioids. An approval under the Notice of Compliance with Conditions means that a product shows potential benefit, possesses high quality and an acceptable safety profile based on a benefit-risk evaluation. Nabiximols is also approved in the United Kingdom and some European countries (e.g., Spain). The United States Food and Drug Administration (FDA) has not yet approved nabiximols as a treatment for any medical condition. Currently it is under investigation by the FDA under the Investigational New Drug Application (IND) for the treatment of cancer pain. Beside THC and CBD, nabiximols also contains other cannabinoids, terpenoids, and flavonoids.

### C. Methods for Making a Composition

In one aspect, disclosed are methods for making a disclosed composition. Thus, in various aspects, the method comprises the step of combining: (a) a cannabinoid in an amount of from about 10 wt % to about 14 wt %; (b) an oil; and (c) a wax, thereby providing the disclosed composition.

In one aspect, disclosed are methods for making a disclosed composition. Thus, in various aspects, the method comprises the step of combining: (a) a cannabinoid in an amount of from about 10 wt % to about 14 wt %; (b) an oil selected from MCT oil and coconut oil; and (c) a wax, thereby providing the disclosed composition.

In a further aspect, the composition comprises an oil. Examples of oils include, but are not limited to, olive oil, almond oil, apricot kernel oil, avocado oil, borage seed oil, camellia seed oil (tea oil), cranberry seed oil, evening primrose oil, grapeseed oil, hazelnut oil, hemp seed oil, jojoba, kukui nut oil, macademia nut oil, meadowfoam oil, peanut oil, pecan oil, pomegranate seed oil, rose hip oil, seabuckthorn berry oil, sesame seed oil, coconut oil, sunflower oil, watermelon seed oil, and MCT oil, or mixtures thereof. Without wishing to be bound by theory, the amount of oil present can be dependent upon the formulation of the composition. For example, in various aspects, the oil is present in an amount of from about 20 wt % to about 30 wt %, 25 wt % to about 30 wt %, or 20 wt % to about 25 wt %. In various further aspects, the oil is present in an amount of from about 50 wt % to about 98 wt %, 60 wt % to about 98 wt %, 70 wt % to about 98 wt %, 80 wt % to about 98 wt %, 90 wt % to about 98 wt %, 50 wt % to about 90 wt %, 50 wt % to about 80 wt %, 50 wt % to about 70 wt %, or 50 wt % to about 60 wt %.

In various aspects, the composition comprises an oil selected from MCT oil and coconut oil. In a further aspect, the oil is MCT oil. In a still further aspect, the oil is coconut oil.

In various aspects, the composition comprises a cannabinoid. In various further aspects, the cannabinoid is THC, CBD, THCA, CBDA, CBN, CBG, CBC, or CBGM, or a mixture thereof. In a further aspect, the cannabinoid is THC or CBD, or a mixture thereof In a still further aspect, the cannabinoid is THC. In yet a further aspect, the cannabinoid is CBD.

In a further aspect, the cannabinoid is present in an amount of from about 10 wt % to about 14 wt %. Thus, in various aspects, the cannabinoid is present in an amount of from about 10 wt % to about 13 wt %, about 10 wt % to about 12 wt %, about 10 wt % to about 11 wt %, about 11 wt % to about 14 wt %, about 12 wt % to about 14 wt %, about 13 wt % to about 14 wt %, or about 11 wt % to about 13 wt %.

In a further aspect, the composition further comprises vitamin E.

In a further aspect, the composition comprises vitamin E in an amount of from about 0.25 wt % to about 4 wt %, about 0.25 wt % to about 3 wt %, about 0.25 wt % to about 2 wt %, about 0.25 wt % to about 1 wt %, about 0.25 wt % to about 0.75 wt %, about 0.75 wt % to about 4 wt %, about 1 wt % to about 4 wt %, about 2 wt % to about 4 wt %, or about 3 wt % to about 4 wt %.

In a further aspect, the composition further comprises one or more of cocoa butter, shea butter, an essential oil, and a jojoba oil. In a still further aspect, the composition further comprises two or more of cocoa butter, shea butter, an essential oil, and a jojoba oil. In yet a further aspect, the composition further comprises three or more of cocoa butter, shea butter, an essential oil, and a jojoba oil. In an even further aspect, the composition further comprises each of cocoa butter, shea butter, an essential oil, and a jojoba oil. In a still further aspect, the composition further comprises cocoa butter. In yet a further aspect, the composition further comprises shea butter. In an even further aspect, the composition further comprises jojoba oil.

In various aspects, vitamin B12 is essentially absent from the composition. In a further aspect, vitamin B12 is present in the composition in an amount less than less than about 10 wt %, less than about 5 wt %, less than about 1 wt %, less than about 0.5 wt %, less than about 0.1 wt %, less than about 0.05 wt %, or less than about 0.01 wt %.

In various aspects, a nonsteroidal anti-inflammatory drug (NSAID) is essentially absent from the composition. In a further aspect, a NSAID is present in the composition in an amount less than less than about 10 wt %, less than about 5 wt %, less than about 1 wt %, less than about 0.5 wt %, less than about 0.1 wt %, less than about 0.05 wt %, or less than about 0.01 wt %.

In a further aspect, the composition further comprises an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others.

In a further aspect, the additive is present in an amount of from about 0.01 wt % to about 10 wt % of the composition. In a still further aspect, the additive is present in an amount of from about 0.01 wt % to about 8 wt % of the composition. In yet a further aspect, the additive is present in an amount of from about 0.01 wt % to about 6 wt % of the composition. In an even further aspect, the additive is present in an amount of from about 0.01 wt % to about 4 wt % of the composition. In a still further aspect, the additive is present in an amount of from about 0.01 wt % to about 2 wt % of the composition. In yet a further aspect, the additive is present in an amount of from about 0.01 wt % to about 1 wt % of the composition. In an even further aspect, the additive is present in an amount of from about 0.1 wt % to about 10 wt % of the composition. In a still further aspect, the additive is present in an amount of from about 1 wt % to about 10 wt % of the composition. In yet a further aspect, the additive is present in an amount of from about 2 wt % to about 10 wt % of the composition. In an even further aspect, the additive is present in an amount of from about 4 wt % to about 10 wt % of the composition. In a still further aspect, the additive is present in an amount of from about 6 wt % to about 10 wt % of the composition. In yet a further aspect, the additive is present in an amount of from about 8 wt % to about 10 wt % of the composition.

### D. Methods of Using the Compositions

The disclosed compositions are useful in treating or ameliorating an injury such as, for example, a muscle injury, a joint injury, a cartilage injury, a tendon injury, or a combination thereof.

The compounds or compositions can be administered to the subject via topical administration. Administration can be continuous or intermittent. A preparation can be administered therapeutically; that is, administered to treat or ameliorate an existing injury.

The therapeutically effective amount or dosage of the composition can vary within wide limits. Such a dosage is adjusted to the individual requirements in each particular case including the specific composition(s) being administered and the condition being treated, as well as the patient being treated. In general, Single dose compositions can contain such amounts or submultiples thereof of the composition to make up the daily dose. The dosage can be adjusted by the individual physician in the event of any contraindications. Dosage can vary, and can be administered in one or more dose administrations daily, for one or several days.

In a further aspect, the subject is a mammal. In a still further aspect, the mammal is a human.

In a further aspect, the injury is selected from a muscle injury, a joint injury, a cartilage injury, a tendon injury, or a combination thereof.
1. Treatment Methods

The compounds disclosed herein are useful for treating or ameliorating injuries such as, for example, muscle injuries, joint injuries, cartilage injuries, and/or tendon injuries. Thus, provided is a method comprising administering a therapeutically effective amount of a disclosed composition to a subject. In a further aspect, the method can be a method for treating an injury.
a. Treating an Injury

In one aspect, disclosed are methods for treating an injury on a subject, the method comprising topically administering to the injury an effective amount of a disclosed composition. Examples of injuries include, but are not limited to, muscle injuries, joint injuries, cartilage injuries, and/or tendon injuries.

Thus, in one aspect, disclosed are methods for treating an injury on a subject, the method comprising topically administering to the injury an effective amount of a composition comprising: (a) a cannabinoid in an amount of from about 10 wt % to about 14 wt %; (b) an oil; and (c) a wax, wherein the injury is a muscle injury, a joint injury, a cartilage injury, a tendon injury, or a combination thereof

In one aspect, disclosed are methods for treating an injury on a subject, the method comprising topically administering to the injury an effective amount of a composition comprising: (a) a cannabinoid in an amount of from about 10 wt % to about 14 wt %; (b) an oil selected from MCT oil and coconut oil; and (c) a wax, wherein the injury is a muscle injury, a joint injury, a cartilage injury, a tendon injury, or a combination thereof

In various aspects, the subject is a mammal. In a further aspect, the subject is a human.

In a further aspect, the method further comprises the step of identifying a subject in need of treatment of the injury. In a still further aspect, the subject has been diagnosed with a need for treatment of the injury prior to the administering step.

In a further aspect, the method further comprises administering at least one agent known for the treatment of pain, wherein the composition and the agent are not co-formulated.

Examples of agents known for the treatment of pain include, but are not limited to, non-steroidal anti-inflammatory drugs (NSAIDs), opioids, counterirritants, salicylates, and capsaicin.

In a further aspect, the agent is a NSAID. Examples of NSADs include, but are not limited to, aspirin, ibuprofen, naproxen, and celecoxib.

In a further aspect, the agent is an opioid. Examples of opioids include, but are not limited to, oxycodone, hydrocodone, codeine, and morphine.

In a further aspect, the agent is a counterirritant. Examples of counterirritants include, but are not limited to, menthol, methyl salicylate, and camphor.

In a further aspect, the agent is a salicylate. Examples of salicylates include, but are not limited to, magnesium salicylate, aspirin, choline salicylate, diflunisal, and salsalate.

In a further aspect, the agent is capsaicin.

In a further aspect, the agent and the composition are administered sequentially. In a still further aspect, the agent and the composition are administered simultaneously.
2. Use of Compositions

In one aspect, the invention relates to the use of a disclosed composition or a product of a disclosed method. In a further aspect, a use relates to the manufacture of a medicament for the treatment of or alleviation of pain related to an injury on a subject.

Also provided are the uses of the disclosed compositions and products. In one aspect, the invention relates to use of at least one disclosed composition. In a further aspect, the composition used is a product of a disclosed method of making.

In a further aspect, the use relates to a process for preparing a pharmaceutical composition comprising a therapeutically effective amount of a disclosed composition or a product of a disclosed method of making, for use as a medicament.

In a further aspect, the use relates to a process for preparing a pharmaceutical composition comprising a therapeutically effective amount of a disclosed composition or a product of a disclosed method of making, wherein a pharmaceutically acceptable carrier is intimately mixed with a therapeutically effective amount of the composition or the product of a disclosed method of making.

In various aspects, the use relates to a treatment of or alleviation of pain related to an injury on a subject. In one aspect, the use is characterized in that the subject is a human. In one aspect, the use is characterized in that the injury is a muscle injury, a joint injury, a cartilage injury, a tendon injury, or a combination thereof.

In a further aspect, the use relates to the manufacture of a medicament for the treatment of or alleviation of pain related to an injury on a subject.

It is understood that the disclosed uses can be employed in connection with the disclosed compositions, products of disclosed methods of making, methods, and kits. In a further aspect, the invention relates to the use of a disclosed composition or a disclosed product in the manufacture of a medicament for the treatment of or alleviation of pain related to an injury on a mammal. In a further aspect, the injury is a muscle injury, a joint injury, a cartilage injury, a tendon injury, or a combination thereof
3. Manufacture of a Medicament

In one aspect, the invention relates to a method for the manufacture of a medicament for treating or alleviating pain related to an injury on a subject, the method comprising combining a therapeutically effective amount of a disclosed composition or product of a disclosed method with a pharmaceutically acceptable carrier or diluent.

As regards these applications, the present method includes the administration to an animal, particularly a mammal, and more particularly a human, of a therapeutically effective amount of the composition effective in the treatment of the injury. The dose administered to an animal, particularly a human, in the context of the present invention should be sufficient to affect a therapeutic response in the animal over a reasonable time frame. One skilled in the art will recognize that dosage will depend upon a variety of factors including the condition of the animal and the body weight of the animal.

The size of the dose also will be determined by the route, timing, and frequency of administration as well as the existence, nature, and extent of any adverse side effects that might accompany the administration of the composition and the desired physiological effect. It will be appreciated by one of skill in the art that various conditions or injuries, in particular chronic conditions or injuries, may require prolonged treatment involving multiple administrations.

Thus, in one aspect, the invention relates to the manufacture of a medicament comprising combining a disclosed composition or a product of a disclosed method of making, with a pharmaceutically acceptable carrier or diluent.

### E. Kits

In one aspect, disclosed are kits comprising a disclosed composition. Thus, in various aspects, disclosed are kits comprising a composition comprising: (a) a cannabinoid in an amount of from about 10 wt % to about 14 wt %; (b) an oil; and (c) a wax, and one or more of: (d) an agent known for the treatment of pain; and (e) instructions for treating an injury selected from a muscle injury, a joint injury, a cartilage injury, a tendon injury, or a combination thereof, wherein the composition and the agent are not co-formulated.

Also disclosed are kits comprising a composition comprising: (a) a cannabinoid in an amount of from about 10 wt % to about 14 wt %; (b) an oil selected from MCT oil and coconut oil; and (c) a wax, and one or more of: (d) an agent known for the treatment of pain; and (e) instructions for treating an injury selected from a muscle injury, a joint injury, a cartilage injury, a tendon injury, or a combination thereof, wherein the composition and the agent are not co-formulated.

In a further aspect, the at least one compound and the at least one agent are co-packaged.

In a further aspect, the kit comprises the composition and the agent known for the treatment of pain. Examples of agents known for the treatment of pain include, but are not limited to, non-steroidal anti-inflammatory drugs (NSAIDs), opioids, counterirritants, salicylates, and capsaicin.

In a further aspect, the agent is a NSAID. Examples of NSADs include, but are not limited to, aspirin, ibuprofen, naproxen, and celecoxib.

In a further aspect, the agent is an opioid. Examples of opioids include, but are not limited to, oxycodone, hydrocodone, codeine, and morphine.

In a further aspect, the agent is a counterirritant. Examples of counterirritants include, but are not limited to, menthol, methyl salicylate, and camphor.

In a further aspect, the agent is a salicylate. Examples of salicylates include, but are not limited to, magnesium salicylate, aspirin, choline salicylate, diflunisal, and salsalate.

In a further aspect, the agent is capsaicin.

In a further aspect, the agent and the composition are administered sequentially. In a still further aspect, the agent and the composition are administered simultaneously.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds, products, and pharmaceutical compositions. It is also understood that the disclosed kits can be employed in connection with the disclosed methods of using.

The previous description of the disclosed aspects is provided to enable any person skilled in the art to make or use the present disclosure. Various modifications to these aspects will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A composition comprising:
(a) a cannabinoid in an amount of from about 10 wt % to about 14 wt %;
(b) an oil; and
(c) a wax.

2. The composition of claim 1, wherein the composition consists essentially of the cannabinoid, the oil, and the wax.

3. The composition of claim 1, wherein the cannabinoid is Δ9-tetrahydrocannabinol (THC), cannabidiol (CBD), or a mixture thereof

4. The composition of claim 1, wherein the oil is MCT oil.

5. The composition of claim 1, wherein the oil is coconut oil.

6. The composition of claim 1, further comprising vitamin E.

7. The composition of claim 1, further comprising one or more of cocoa butter, shea butter, an essential oil, and a jojoba oil.

8. The composition of claim 1, wherein vitamin B12 is essentially absent from the composition.

9. The composition of claim 1, wherein a nonsteroidal anti-inflammatory drug (NSAID) is essentially absent from the composition.

10. The composition of claim 1, wherein the composition is a topical composition.

11. A method for treating an injury on a subject, the method comprising topically administering to the injury an effective amount of the composition of claim 1, wherein the injury is a muscle injury, a joint injury, a cartilage injury, a tendon injury, or a combination thereof.

12. The method of claim 11, wherein the subject is a mammal.

13. The method of claim 11, wherein the subject is a human.

14. The method of claim 11, further comprising the step of identifying a subject in need of treatment of the injury.

15. The method of claim 11, wherein the subject has been diagnosed with a need for treatment of the injury.

16. The method of claim 11, further comprising administering at least one agent known for the treatment of pain, wherein the composition and the agent are not co-formulated.

17. The method of claim 16, wherein the agent is selected from a NSAID, an opioid, a counterirritant, a salicylate, and capsaicin.

18. A kit comprising the composition of claim 1, and one or more of:
(a) an agent known for the treatment of pain; and
(b) instructions for treating an injury selected from a muscle injury, a joint injury, a cartilage injury, a tendon injury, or a combination thereof,
wherein the composition and the agent are not co-formulated.

19. The kit of claim 18, wherein the agent is selected from a NSAID, an opioid, a counterirritant, a salicylate, and capsaicin.

20. The kit of claim 19, wherein the NSAID is selected from aspirin, ibuprofen, naproxen, and celecoxib.
